SEQUENCE LISTING (12 sequences)

SEQ ID NO: 1 - DNA, 2619 bp, synthetic construct
  source 1..2619: mol_type = other DNA
gctagcgccaccatgttcgtgttcctggtcctgctgccactggtgtccagagtgcagcctacagaaagcatcgtgcggttccccaatatcacaaacctgtgtcctttcggcgaggtgttcaacgccaccagattcgccagcgtgtacgcctggaatagaaagcggatttctaactgtgtggccgactacagcgttctgtacaactctgcctctttcagcaccttcaagtgctacggcgtgtcccctacaaagctcaacgacctgtgcttcaccaacgtgtacgccgactctttcgtgatcagaggggacgaggtgaggcagatcgcccctggccagaccggcaagatcgccgattacaactacaagctgccagatgacttcaccggctgtgtgattgcttggaacagcaacaacctggacagcaaggtgggcggcaattacaattatctgtatagactgttccggaagtccaacctgaagcccttcgagagagatatcagcaccgagatctaccaggcgggctctaccccatgcaacggcgtggaaggatttaactgctacttccctctgcaatcttacggcttccagcctaccaatggcgtaggctaccagccctacagagtggtggtgctgtcttttgagctgttgcacgcccctgctaccgtgtgcggccctaagaagtccacaaacctggtgaaaaacaaatgcgtgaacttcggcggaagcggcggaagcggcggaagcggcggcagcggaggcggcagcactattgaggaacaggccaagacctttctggacaaattcaaccacgaggccgaagacctgttctaccagagcagcctggccagctggaactacaacacaaacatcaccgaggaaaacgtgcagaacatgaacaacgccggagacaagtggtccgccttcctgaaggaacagagcacactggcacagatgtaccccctgcaggagatccaaaacctgaccgtgaagctgcagctgcaggccttgcagcagaacggcagcagcgtgctgtcagaagataagagcaagagactgaatactatcctgaacaccatgagcaccatctactccacaggcaaggtgtgtaatcctgacaacccccaggagtgcctgttgctggagcctggccttaacgagatcatggctaacagcctcgactacaacgaaagactgtgggcctgggaaagctggaggagcgaggtgggaaagcagctgagacctctgtacgaggagtacgtggtcctgaagaacgagatggccagagccaaccactacgaggattacggagactattggcggggcgactacgaggtgaatggcgtggacggctatgactacagccggggacaactgatcgaggacgttgaacacacattcgaggaaatcaagcccctgtatgaacacctccatgcttacgtgagagccaaactgatgaacgcctaccctagctacatctcccccatcggctgtctgcctgcccacctgctgggcgatatgtggggcagattctggacaaacctgtacagcctgaccgtgccttttggccagaaacccaacatcgacgtgaccgacgccatggtcgaccaagcctgggatgcgcagagaatcttcaaagaggccgagaagttcttcgtgtccgtgggcctgcccaatatgacccagggcttctgggaaaacagtatgctgacagatcctggcaatgtccagaaggccgtgtgccaccctacagcctgggacctgggcaagggagatttcagaatcctgatgtgcaccaaggtgaccatggacgacttcctgaccgcccatcacgagatgggccacatccagtacgacatggcttacgccgcccagcctttcctgctgcggaatggcgccaatgaaggcttccacgaggccgtgggcgaaatcatgtctctgagcgctgctacccccaagcacctgaaatctatcggcctgctgagcccagatttccaagaggataatgaaacagagatcaactttctgctgaaacaggccctgaccatcgtgggaacactgcctttcacctacatgctggaaaagtggcggtggatggtgttcaagggcgagatccccaaggaccagtggatgaaaaagtggtgggagatgaagcgggaaatcgtgggagtggttgagcctgtgccccacgacgagacatactgcgaccctgccagcctgtttcacgtctctaatgattactcctttatccgctactacacccggaccctgtatcaatttcagttccaggaggccctgtgccaggccgctaagcacgaaggccccctccacaagtgcgatatcagcaacagcacagaggctggccagaagctgttcaacatgctgagactgggtaagagcgagccttggacgctggccctggaaaacgtggtgggcgctaaaaacatgaacgttagacctttacttaattacttcgagcctctgtttacatggctgaaagatcagaacaagaacagctttgtgggctggagcaccgattggtctccttacgccgaccagagcatcaaggtcagaatcagcctgaagagcgccctgggcgacaaggcctactgataactcgag

SEQ ID NO: 2 - protein, 865 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSGGSGGSGGSGGGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAY

SEQ ID NO: 3 - DNA, 4047 bp, synthetic construct
  source 1..4047: mol_type = other DNA
gctagcgccaccatgttcgtgttcctggtgctgctgccactggtgtcccgggtgcagcctacagagagcatcgtgcgcttccctaacatcacaaacctgtgccctttcggcgaggtgtttaatgccaccagatttgccagcgtatacgcctggaaccggaagagaatctctaactgcgtagctgactactctgtgctgtataacagcgcctcctttagcacattcaagtgctacggcgtgtctcccaccaaactgaacgatctgtgcttcacaaacgtgtacgccgacagcttcgttatcaggggcgatgaggtgagacaaatcgcccctggacaaacaggaaagatcgccgattacaactataagctgcctgacgatttcacaggctgcgtcatcgcctggaacagcaataatctggacagcaaggtgggcggcaactacaactacctgtaccggctgttcagaaagtctaacctgaaacctttcgagcgtgacatttccaccgagatctatcaggccggctccaccccttgtaacggcgtcgaaggatttaactgctacttccccttgcagagctacggcttccagcctacctacggcgtgggttaccaaccttatagagttgttgtgctgtctttcgaactgctgcacgcccctgccaccgtctgtggcccaaagaagagcactaatttggtgaaaaacaagtgcgtgaacttcggcggcagcggcggatctggcggctcaggcggcagcggcggaggtagagtgcagcctaccgagagcatcgtgcgcttcccgaacatcaccaatctgtgccctttcggcgaggtgttcaacgccacccggttcgccagcgtctacgcctggaacagaaagagaatcagcaactgcgtggccgattattctgtgctgtataactcagcctcgttttctacattcaagtgctacggcgtgtcccccaccaagctgaatgacctgtgttttaccaacgtgtatgccgacagcttcgtgatcagaggagacgaggtacgacagatcgctccaggccagaccggcaacatcgctgattacaactacaagctgcctgatgacttcaccggctgtgtgatcgcttggaacagcaacaacctggactccaaggtgggcggcaactacaactacctgtaccggctgttcagaaagagcaacctgaagccttttgagagagatatcagcaccgagatctaccaagccggcagcactccctgcaacggcgtgaagggcttcaactgttatttcccgctgcagagctacggcttccagcctacctacggcgtgggataccagccctacagagtagtggtactgagctttgaactgctccatgcccccgccactgtttgtggccccaaaaagtccaccaacctcgtgaaaaacaagtgcgtgaatttcggcggcagcgggggcagcggaggcagcggcggctcaggcggcggcagagtgcagcctacagaaagcatcgtgcggttcccaaacatcaccaacctgtgtccttttggcgaagtgttcaacgccacaagatttgccagcgtgtacgcctggaatagaaaacgcattagtaattgtgttgctgactacagcgtgctgtacaacagcgcctctttcagcacctttaagtgctacggcgtttctcccaccaagctgaatgatctctgtttcacaaacgtgtacgccgatagcttcgtgatcagaggggacgaggtgagacagatcgccccaggacagaccggcaccatcgccgattacaactacaaactgcccgacgattttaccggctgcgttatagcttggaactctaacaatctcgatagcaaggtgggcggaaattacaactacttgtacagactgttcagaaagagcaacctgaagcctttcgagcgcgacatcagcaccgaaatctaccaggctggctccacaccttgcaatggcgtgaaaggcttcaactgctacttccctctgcaatcttacgggttccagcccacctacggagtcggataccagccttacagagtggtggtgctgagcttcgaactgctgcatgcccctgccacagtgtgcggccctaagaagagcacaaaccttgtgaagaataagtgcgtgaatttcggcggcagcggcgggagcggaggttccggcggctctggaggcggaagcaccatcgaggaacaggcaaagaccttcctagacaaattcaaccacgaggccgaggacctgttctaccagagcagcctggcctcctggaactacaatacgaacatcacggaagagaacgtccagaacatgaacaacgccggcgacaagtggagcgcctttctgaaggaacaaagcaccctggcccagatgtaccccctgcaggagatccaaaatctgaccgtgaagctgcagctgcaggcgctgcagcagaacggctccagcgtgctgtctgaggacaagagcaagagactgaacaccatcctgaataccatgagcaccatctacagcaccggcaaagtgtgcaatcctgacaacccccaggagtgcctgttactggagcctggcctgaacgagattatggccaacagcctggactacaacgagcggctgtgggcctgggagagctggcggtccgaggtgggcaagcagctgcggcctctgtacgaggaatacgtggtgctgaagaacgaaatggccagagccaatcactacgaggattacggcgactactggagaggcgactacgaagtgaacggcgtggacggctacgactacagcagaggccagctgatcgaggacgtggaacacaccttcgaagaaatcaagcctctgtatgaacacctgcacgcctatgtgcgggccaaactgatgaacgcttacccttcctacatcagccctatcggttgcctgcctgctcacctgctgggcgacatgtggggtagattctggaccaacctgtactccctgaccgtgcccttcggccagaaacccaacatcgacgtgaccgacgccatggtcgaccaggcctgggatgcccagaggatcttcaaagaggctgaaaagttcttcgtgagcgtgggactgcctaacatgacccagggcttctgggaaaacagcatgctgacagatcctggcaacgtgcagaaagccgtgtgccacccaacggcctgggacctgggcaagggcgattttagaatcctgatgtgcacaaaggtgacaatggacgacttcctgaccgcccaccacgagatgggccacatccagtacgacatggcctacgccgctcagcccttcctgctgcggaatggagccaacgagggcttccacgaagccgtgggcgaaatcatgtctctgagtgccgccacaccaaagcacctgaagtctatcggcctgctgagccctgatttccaggaagataacgagacagagatcaacttcctcctgaagcaggccctgacaatcgtggggaccctgcccttcacctacatgctggaaaagtggcgttggatggtgttcaagggcgagatccctaaagatcagtggatgaagaagtggtgggagatgaagcgggaaatcgtgggagtggtggaacctgtgccccacgacgagacatactgcgaccccgcatctctattccacgtgtctaacgactacagcttcatccggtactacaccagaaccctgtaccagttccaatttcaggaggcactgtgccaggccgccaagcacgagggccctctgcacaagtgtgacatcagcaactctacagaggccggtcagaagctgttcaacatgctgagactgggaaagagcgagccttggactctggctctggagaatgtggtcggggccaagaacatgaatgtcagacctctgctgaactacttcgaacctctatttacatggctgaaggaccagaacaaaaattctttcgtcgggtggagtaccgactggtctccttacgccgaccagagcattaaggtgcggatttcccttaagtcagccctgggcgacaaagcctactgataactcgag

SEQ ID NO: 4 - protein, 1024 aa, synthetic construct
MFVFLVLLPLVSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSGGSGGSGGSGGGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYGGSGGSGGSGGSGGGARPKQRRPQGLPNNTASWFTALTQATEGALNTPKDHIGTRNGDAALALLLLDRLNQLESKMGDQELIRQGTDYKHWPQIAELAPSASAFFGMSRIGMEVTPSGTWLTYTGFKDQLILLNENIDAYKTFPPTEPKKDKKKKADET

SEQ ID NO: 5 - DNA, 3096 bp, synthetic construct
  source 1..3096: mol_type = other DNA
gctagcgccaccatgttcgtgttcctggtgctgctgcctctggtgtctagagtgcagccgaccgaatctatcgtgcggttccctaacatcaccaacctgtgtccttttggcgaggtgtttaacgccacccggttcgccagcgtgtacgcctggaatagaaagcggatcagcaactgcgtggccgattacagcgtgctgtacaacagcgcttctttctccacatttaagtgctacggcgtatcccctacaaagctgaatgatctgtgcttcaccaacgtttacgccgattccttcgtcatccggggcgacgaggtaagacaaatcgcccctggccaaacaggcaagatcgccgactacaactacaagctgcccgacgatttcaccggctgtgtgatcgcttggaacagcaacaatctggacagcaaagtggggggcaactacaactacctgtacagactgttcagaaaaagcaatctgaaaccatttgaacgggacataagcaccgagatctaccaggccggctccacaccttgtaatggtgtggaaggcttcaactgctacttccctctgcagagctacggctttcagcctaccaacggcgtcggataccagccttaccgggtggtggtgctgtcctttgaactgcttcacgcccctgctaccgtgtgcggccccaaaaaaagcaccaatctggtgaagaacaagtgtgtgaactttggaggcagcggcggctctggcggctccggcggctcgggcggcggcagcaccatcgaggaacaggccaagacattcctcgacaaattcaaccacgaagccgaggacctgttctaccagtctagcctggcctcttggaactacaacaccaacatcaccgaggaaaacgtgcagaacatgaacaacgccggcgacaaatggtctgctttcctcaaggaacagagcaccctggcccaaatgtaccccctgcaggagatccaaaacctgacagtgaagctgcagctgcaggccctgcagcagaatggctcttctgtgctgagcgaggacaagtccaagcgtctgaacaccatcctgaacacaatgagcaccatctacagcaccggcaaggtgtgcaaccccgacaacccccaggagtgcctgctgctggaacctggcctgaacgagatcatggctaacagcctggactacaacgagagactctgggcctgggaatcttggcgcagcgaggtgggcaagcagctgcggcccctctatgaagagtacgtcgtgctgaagaacgagatggccagagccaatcactacgaggactacggcgactattggaggggtgactacgaggttaacggcgtggacggctacgactactctagaggacaactgattgaggatgtggaacacacattcgaagagatcaagcccctgtacgaacacctgcacgcctacgtgcgggctaagctgatgaacgcctaccctagctacatctctcctatcggctgcctgcctgcccacctgctgggcgacatgtggggcagattctggacaaatctgtactccctgactgtaccttttggccagaaacccaatatcgacgtgaccgacgccatggttgaccaggcctgggatgcccagagaatcttcaaagaggccgagaagttcttcgtgtctgtgggactgccaaatatgacccagggcttctgggagaacagcatgctgacagaccctggaaacgtgcagaaagctgtgtgtcatcctaccgcctgggacctgggcaaaggcgactttagaatcctcatgtgcaccaaggtgacaatggacgatttcctgaccgcacatcacgagatgggccacatccagtacgatatggcttatgccgcccagccgtttctgctgcggaacggcgccaacgagggcttccacgaagccgtgggagagatcatgagcctgagcgccgcaaccccaaagcacctgaagtctatcggactgctgtcccctgacttccaggaggataatgagacagaaatcaacttcctgctcaagcaggccttgaccatagtgggcaccctgcctttcacatacatgctggagaaatggagatggatggtgttcaagggcgaaatccctaaggaccagtggatgaagaagtggtgggagatgaagcgggaaatcgtgggggtggtcgagcctgtgccccacgacgagacatattgcgatcccgccagcctgttccacgtgagcaacgattacagcttcatcagatactacacaagaaccctgtaccagttccagttccaggaggccctgtgccaggccgccaagcacgagggacctctgcacaagtgcgacatcagcaattccaccgaggccggacagaagctgttcaacatgctgagattaggaaagtctgaaccttggaccctcgccctggaaaacgtggtgggcgctaagaacatgaacgtgcggccactgcttaactacttcgaacctctgtttacctggctgaaggatcaaaacaagaacagctttgttggctggtccaccgactggagcccctacgccgaccagagcattaaggtgagaatcagcctgaagagcgccctgggcgataaggcctatggcggcagcggcggtagcggtggcagcggagggagcggcggcggcgccagacctaagcagcggagaccccagggcctgcctaacaacaccgccagctggttcacagccctgacacaggctaccgagggcgccctgaatactccaaaggatcacatcggaacaagaaacggcgatgctgccctggctctgctgctgctggacagactgaaccagctggaaagcaagatgggcgaccaggagctgatcagacagggcacagactacaagcactggcctcaaatcgccgaactggctcctagcgccagcgccttcttcggaatgagcagaatcggcatggaagtcacccccagcggaacctggctgacctacaccggcttcaaggaccagctgatcctgctgaacgagaatatcgacgcctataagaccttccccccgaccgagcctaaaaaggataagaaaaagaaggccgatgagacatgataactcgag

SEQ ID NO: 6 - protein, 1024 aa, synthetic construct; identical to SEQ ID NO: 4

SEQ ID NO: 7 - DNA, 2559 bp, synthetic construct
  source 1..2559: mol_type = other DNA
agagtgcagcctacagaaagcatcgtgcggttccccaatatcacaaacctgtgtcctttcggcgaggtgttcaacgccaccagattcgccagcgtgtacgcctggaatagaaagcggatttctaactgtgtggccgactacagcgttctgtacaactctgcctctttcagcaccttcaagtgctacggcgtgtcccctacaaagctcaacgacctgtgcttcaccaacgtgtacgccgactctttcgtgatcagaggggacgaggtgaggcagatcgcccctggccagaccggcaagatcgccgattacaactacaagctgccagatgacttcaccggctgtgtgattgcttggaacagcaacaacctggacagcaaggtgggcggcaattacaattatctgtatagactgttccggaagtccaacctgaagcccttcgagagagatatcagcaccgagatctaccaggcgggctctaccccatgcaacggcgtggaaggatttaactgctacttccctctgcaatcttacggcttccagcctaccaatggcgtaggctaccagccctacagagtggtggtgctgtcttttgagctgttgcacgcccctgctaccgtgtgcggccctaagaagtccacaaacctggtgaaaaacaaatgcgtgaacttcggcggaagcggcggaagcggcggaagcggcggcagcggaggcggcagcactattgaggaacaggccaagacctttctggacaaattcaaccacgaggccgaagacctgttctaccagagcagcctggccagctggaactacaacacaaacatcaccgaggaaaacgtgcagaacatgaacaacgccggagacaagtggtccgccttcctgaaggaacagagcacactggcacagatgtaccccctgcaggagatccaaaacctgaccgtgaagctgcagctgcaggccttgcagcagaacggcagcagcgtgctgtcagaagataagagcaagagactgaatactatcctgaacaccatgagcaccatctactccacaggcaaggtgtgtaatcctgacaacccccaggagtgcctgttgctggagcctggccttaacgagatcatggctaacagcctcgactacaacgaaagactgtgggcctgggaaagctggaggagcgaggtgggaaagcagctgagacctctgtacgaggagtacgtggtcctgaagaacgagatggccagagccaaccactacgaggattacggagactattggcggggcgactacgaggtgaatggcgtggacggctatgactacagccggggacaactgatcgaggacgttgaacacacattcgaggaaatcaagcccctgtatgaacacctccatgcttacgtgagagccaaactgatgaacgcctaccctagctacatctcccccatcggctgtctgcctgcccacctgctgggcgatatgtggggcagattctggacaaacctgtacagcctgaccgtgccttttggccagaaacccaacatcgacgtgaccgacgccatggtcgaccaagcctgggatgcgcagagaatcttcaaagaggccgagaagttcttcgtgtccgtgggcctgcccaatatgacccagggcttctgggaaaacagtatgctgacagatcctggcaatgtccagaaggccgtgtgccaccctacagcctgggacctgggcaagggagatttcagaatcctgatgtgcaccaaggtgaccatggacgacttcctgaccgcccatcacgagatgggccacatccagtacgacatggcttacgccgcccagcctttcctgctgcggaatggcgccaatgaaggcttccacgaggccgtgggcgaaatcatgtctctgagcgctgctacccccaagcacctgaaatctatcggcctgctgagcccagatttccaagaggataatgaaacagagatcaactttctgctgaaacaggccctgaccatcgtgggaacactgcctttcacctacatgctggaaaagtggcggtggatggtgttcaagggcgagatccccaaggaccagtggatgaaaaagtggtgggagatgaagcgggaaatcgtgggagtggttgagcctgtgccccacgacgagacatactgcgaccctgccagcctgtttcacgtctctaatgattactcctttatccgctactacacccggaccctgtatcaatttcagttccaggaggccctgtgccaggccgctaagcacgaaggccccctccacaagtgcgatatcagcaacagcacagaggctggccagaagctgttcaacatgctgagactgggtaagagcgagccttggacgctggccctggaaaacgtggtgggcgctaaaaacatgaacgttagacctttacttaattacttcgagcctctgtttacatggctgaaagatcagaacaagaacagctttgtgggctggagcaccgattggtctccttacgccgaccagagcatcaaggtcagaatcagcctgaagagcgccctgggcgacaaggcctac

SEQ ID NO: 8 - protein, 854 aa, synthetic construct
SRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSGGSGGSGGSGGGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAY

SEQ ID NO: 9 - DNA, 3987 bp, synthetic construct
  source 1..3987: mol_type = other DNA
cgggtgcagcctacagagagcatcgtgcgcttccctaacatcacaaacctgtgccctttcggcgaggtgtttaatgccaccagatttgccagcgtatacgcctggaaccggaagagaatctctaactgcgtagctgactactctgtgctgtataacagcgcctcctttagcacattcaagtgctacggcgtgtctcccaccaaactgaacgatctgtgcttcacaaacgtgtacgccgacagcttcgttatcaggggcgatgaggtgagacaaatcgcccctggacaaacaggaaagatcgccgattacaactataagctgcctgacgatttcacaggctgcgtcatcgcctggaacagcaataatctggacagcaaggtgggcggcaactacaactacctgtaccggctgttcagaaagtctaacctgaaacctttcgagcgtgacatttccaccgagatctatcaggccggctccaccccttgtaacggcgtcgaaggatttaactgctacttccccttgcagagctacggcttccagcctacctacggcgtgggttaccaaccttatagagttgttgtgctgtctttcgaactgctgcacgcccctgccaccgtctgtggcccaaagaagagcactaatttggtgaaaaacaagtgcgtgaacttcggcggcagcggcggatctggcggctcaggcggcagcggcggaggtagagtgcagcctaccgagagcatcgtgcgcttcccgaacatcaccaatctgtgccctttcggcgaggtgttcaacgccacccggttcgccagcgtctacgcctggaacagaaagagaatcagcaactgcgtggccgattattctgtgctgtataactcagcctcgttttctacattcaagtgctacggcgtgtcccccaccaagctgaatgacctgtgttttaccaacgtgtatgccgacagcttcgtgatcagaggagacgaggtacgacagatcgctccaggccagaccggcaacatcgctgattacaactacaagctgcctgatgacttcaccggctgtgtgatcgcttggaacagcaacaacctggactccaaggtgggcggcaactacaactacctgtaccggctgttcagaaagagcaacctgaagccttttgagagagatatcagcaccgagatctaccaagccggcagcactccctgcaacggcgtgaagggcttcaactgttatttcccgctgcagagctacggcttccagcctacctacggcgtgggataccagccctacagagtagtggtactgagctttgaactgctccatgcccccgccactgtttgtggccccaaaaagtccaccaacctcgtgaaaaacaagtgcgtgaatttcggcggcagcgggggcagcggaggcagcggcggctcaggcggcggcagagtgcagcctacagaaagcatcgtgcggttcccaaacatcaccaacctgtgtccttttggcgaagtgttcaacgccacaagatttgccagcgtgtacgcctggaatagaaaacgcattagtaattgtgttgctgactacagcgtgctgtacaacagcgcctctttcagcacctttaagtgctacggcgtttctcccaccaagctgaatgatctctgtttcacaaacgtgtacgccgatagcttcgtgatcagaggggacgaggtgagacagatcgccccaggacagaccggcaccatcgccgattacaactacaaactgcccgacgattttaccggctgcgttatagcttggaactctaacaatctcgatagcaaggtgggcggaaattacaactacttgtacagactgttcagaaagagcaacctgaagcctttcgagcgcgacatcagcaccgaaatctaccaggctggctccacaccttgcaatggcgtgaaaggcttcaactgctacttccctctgcaatcttacgggttccagcccacctacggagtcggataccagccttacagagtggtggtgctgagcttcgaactgctgcatgcccctgccacagtgtgcggccctaagaagagcacaaaccttgtgaagaataagtgcgtgaatttcggcggcagcggcgggagcggaggttccggcggctctggaggcggaagcaccatcgaggaacaggcaaagaccttcctagacaaattcaaccacgaggccgaggacctgttctaccagagcagcctggcctcctggaactacaatacgaacatcacggaagagaacgtccagaacatgaacaacgccggcgacaagtggagcgcctttctgaaggaacaaagcaccctggcccagatgtaccccctgcaggagatccaaaatctgaccgtgaagctgcagctgcaggcgctgcagcagaacggctccagcgtgctgtctgaggacaagagcaagagactgaacaccatcctgaataccatgagcaccatctacagcaccggcaaagtgtgcaatcctgacaacccccaggagtgcctgttactggagcctggcctgaacgagattatggccaacagcctggactacaacgagcggctgtgggcctgggagagctggcggtccgaggtgggcaagcagctgcggcctctgtacgaggaatacgtggtgctgaagaacgaaatggccagagccaatcactacgaggattacggcgactactggagaggcgactacgaagtgaacggcgtggacggctacgactacagcagaggccagctgatcgaggacgtggaacacaccttcgaagaaatcaagcctctgtatgaacacctgcacgcctatgtgcgggccaaactgatgaacgcttacccttcctacatcagccctatcggttgcctgcctgctcacctgctgggcgacatgtggggtagattctggaccaacctgtactccctgaccgtgcccttcggccagaaacccaacatcgacgtgaccgacgccatggtcgaccaggcctgggatgcccagaggatcttcaaagaggctgaaaagttcttcgtgagcgtgggactgcctaacatgacccagggcttctgggaaaacagcatgctgacagatcctggcaacgtgcagaaagccgtgtgccacccaacggcctgggacctgggcaagggcgattttagaatcctgatgtgcacaaaggtgacaatggacgacttcctgaccgcccaccacgagatgggccacatccagtacgacatggcctacgccgctcagcccttcctgctgcggaatggagccaacgagggcttccacgaagccgtgggcgaaatcatgtctctgagtgccgccacaccaaagcacctgaagtctatcggcctgctgagccctgatttccaggaagataacgagacagagatcaacttcctcctgaagcaggccctgacaatcgtggggaccctgcccttcacctacatgctggaaaagtggcgttggatggtgttcaagggcgagatccctaaagatcagtggatgaagaagtggtgggagatgaagcgggaaatcgtgggagtggtggaacctgtgccccacgacgagacatactgcgaccccgcatctctattccacgtgtctaacgactacagcttcatccggtactacaccagaaccctgtaccagttccaatttcaggaggcactgtgccaggccgccaagcacgagggccctctgcacaagtgtgacatcagcaactctacagaggccggtcagaagctgttcaacatgctgagactgggaaagagcgagccttggactctggctctggagaatgtggtcggggccaagaacatgaatgtcagacctctgctgaactacttcgaacctctatttacatggctgaaggaccagaacaaaaattctttcgtcgggtggagtaccgactggtctccttacgccgaccagagcattaaggtgcggatttcccttaagtcagccctgggcgacaaagcctac

SEQ ID NO: 10 - protein, 1329 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFGGSGGSGGSGGSGGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGSGGSGGSGGSGGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGSGGSGGSGGSGGGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAY

SEQ ID NO: 11 - DNA, 3036 bp, synthetic construct
  source 1..3036: mol_type = other DNA
agagtgcagccgaccgaatctatcgtgcggttccctaacatcaccaacctgtgtccttttggcgaggtgtttaacgccacccggttcgccagcgtgtacgcctggaatagaaagcggatcagcaactgcgtggccgattacagcgtgctgtacaacagcgcttctttctccacatttaagtgctacggcgtatcccctacaaagctgaatgatctgtgcttcaccaacgtttacgccgattccttcgtcatccggggcgacgaggtaagacaaatcgcccctggccaaacaggcaagatcgccgactacaactacaagctgcccgacgatttcaccggctgtgtgatcgcttggaacagcaacaatctggacagcaaagtggggggcaactacaactacctgtacagactgttcagaaaaagcaatctgaaaccatttgaacgggacataagcaccgagatctaccaggccggctccacaccttgtaatggtgtggaaggcttcaactgctacttccctctgcagagctacggctttcagcctaccaacggcgtcggataccagccttaccgggtggtggtgctgtcctttgaactgcttcacgcccctgctaccgtgtgcggccccaaaaaaagcaccaatctggtgaagaacaagtgtgtgaactttggaggcagcggcggctctggcggctccggcggctcgggcggcggcagcaccatcgaggaacaggccaagacattcctcgacaaattcaaccacgaagccgaggacctgttctaccagtctagcctggcctcttggaactacaacaccaacatcaccgaggaaaacgtgcagaacatgaacaacgccggcgacaaatggtctgctttcctcaaggaacagagcaccctggcccaaatgtaccccctgcaggagatccaaaacctgacagtgaagctgcagctgcaggccctgcagcagaatggctcttctgtgctgagcgaggacaagtccaagcgtctgaacaccatcctgaacacaatgagcaccatctacagcaccggcaaggtgtgcaaccccgacaacccccaggagtgcctgctgctggaacctggcctgaacgagatcatggctaacagcctggactacaacgagagactctgggcctgggaatcttggcgcagcgaggtgggcaagcagctgcggcccctctatgaagagtacgtcgtgctgaagaacgagatggccagagccaatcactacgaggactacggcgactattggaggggtgactacgaggttaacggcgtggacggctacgactactctagaggacaactgattgaggatgtggaacacacattcgaagagatcaagcccctgtacgaacacctgcacgcctacgtgcgggctaagctgatgaacgcctaccctagctacatctctcctatcggctgcctgcctgcccacctgctgggcgacatgtggggcagattctggacaaatctgtactccctgactgtaccttttggccagaaacccaatatcgacgtgaccgacgccatggttgaccaggcctgggatgcccagagaatcttcaaagaggccgagaagttcttcgtgtctgtgggactgccaaatatgacccagggcttctgggagaacagcatgctgacagaccctggaaacgtgcagaaagctgtgtgtcatcctaccgcctgggacctgggcaaaggcgactttagaatcctcatgtgcaccaaggtgacaatggacgatttcctgaccgcacatcacgagatgggccacatccagtacgatatggcttatgccgcccagccgtttctgctgcggaacggcgccaacgagggcttccacgaagccgtgggagagatcatgagcctgagcgccgcaaccccaaagcacctgaagtctatcggactgctgtcccctgacttccaggaggataatgagacagaaatcaacttcctgctcaagcaggccttgaccatagtgggcaccctgcctttcacatacatgctggagaaatggagatggatggtgttcaagggcgaaatccctaaggaccagtggatgaagaagtggtgggagatgaagcgggaaatcgtgggggtggtcgagcctgtgccccacgacgagacatattgcgatcccgccagcctgttccacgtgagcaacgattacagcttcatcagatactacacaagaaccctgtaccagttccagttccaggaggccctgtgccaggccgccaagcacgagggacctctgcacaagtgcgacatcagcaattccaccgaggccggacagaagctgttcaacatgctgagattaggaaagtctgaaccttggaccctcgccctggaaaacgtggtgggcgctaagaacatgaacgtgcggccactgcttaactacttcgaacctctgtttacctggctgaaggatcaaaacaagaacagctttgttggctggtccaccgactggagcccctacgccgaccagagcattaaggtgagaatcagcctgaagagcgccctgggcgataaggcctatggcggcagcggcggtagcggtggcagcggagggagcggcggcggcgccagacctaagcagcggagaccccagggcctgcctaacaacaccgccagctggttcacagccctgacacaggctaccgagggcgccctgaatactccaaaggatcacatcggaacaagaaacggcgatgctgccctggctctgctgctgctggacagactgaaccagctggaaagcaagatgggcgaccaggagctgatcagacagggcacagactacaagcactggcctcaaatcgccgaactggctcctagcgccagcgccttcttcggaatgagcagaatcggcatggaagtcacccccagcggaacctggctgacctacaccggcttcaaggaccagctgatcctgctgaacgagaatatcgacgcctataagaccttccccccgaccgagcctaaaaaggataagaaaaagaaggccgatgagaca

SEQ ID NO: 12 - protein, 1012 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSGGSGGSGGSGGGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYGGSGGSGGSGGSGGGARPKQRRPQGLPNNTASWFTALTQATEGALNTPKDHIGTRNGDAALALLLLDRLNQLESKMGDQELIRQGTDYKHWPQIAELAPSASAFFGMSRIGMEVTPSGTWLTYTGFKDQLILLNENIDAYKTFPPTEPKKDKKKKADET

(19) United States
(12) Patent Application Publication
(10) Pub. No.: US 2025/0249088 A1
(43) Pub. Date: Aug. 7, 2025

(54) THERAPEUTIC AND VACCINE CANDIDATES AGAINST SARS-CoV-2

(71) Applicant: GENEONE LIFE SCIENCE, INC., Seoul (KR)

(72) Inventor: Kar MUTHUMANI, Collegeville, PA (US)

(73) Assignee: GENEONE LIFE SCIENCE, INC., Seoul (KR)

(21) Appl. No.: 18/702,303

(22) PCT Filed: Oct. 31, 2022
(86) PCT No.: PCT/IB2022/060499

§ 371 Date: Apr. 17, 2024

Related U.S. Application Data
(60) Provisional application No. 63/263,311, filed on Oct. 29, 2021.

(51) Int. Cl. A61K 39/215; A61K 39/00; A61K 39/39; A61K 41/00; A61P 37/04; C07K 14/005

(52) U.S. Cl. CPC A61K 39/215; A61K 39/39; A61K 41/0047; A61P 37/04; C07K 14/005; A61K 2039/5256

(57) ABSTRACT

Provided herein is an immunogenic composition comprising a synthetic antigen to COVID spike proteins, particularly the chimeric intermediate structure C-A Complex. Also disclosed herein is a method of preventing and/or treating a COVID infection in a subject in need thereof, by administering the immunogenic composition to the subject.

### CROSS-REFERENCE TO RELATED APPLICATIONS

This application claims priority to U.S. Provisional Application No. 63/263,311, filed Oct. 29, 2021, which is hereby incorporated by reference in its entirety.

### TECHNICAL FIELD

The present invention relates to nucleic acid and protein molecules capable of generating an immune response in a subject.

### BACKGROUND OF THE INVENTION

The SARS-CoV-2 pandemic has caused significant social disruption and economic hardship globally, with an outsized effect in low and middle-income countries. Availability of vaccines against SARS-CoV-2 in such countries is a challenge due to cost and cold chain requirements. Furthermore, the emergence of the B.1.1.7-UK, Brazilian-P1 and South African-B.1.351 strains threaten vaccination program success and raise the potential for new waves of infection.

Updated approaches are needed to rapidly respond to new emerging diseases, especially early in the epidemic when prompt public health intervention strategies can limit mortality and epidemic spread. In particular, emerging respiratory coronaviruses offer a considerable threat to the health of global populations and the economy. Coronaviruses constitute a group of phylogenetically diverse enveloped viruses that encode the largest plus strand RNA genomes and replicate efficiently in most mammals. Human CoV (hCoVs-229e, OC43, NL63, and HKU1) infections typically result in mild to severe upper and lower respiratory tract disease. Severe acute respiratory syndrome coronavirus (SARS-COV) emerged in 2002-2003 causing acute respiratory distress syndrome (ARDS) with 10% mortality overall and up to 50% mortality in aged individuals. Middle Eastern respiratory syndrome coronavirus (MERS-CoV) emerged in the middle east in April of 2012, manifesting as severe pneumonia, acute respiratory distress syndrome (ARDS) and acute renal failure. The virus is still circulating and has been shown to have a mortality rate of ˜49%. Platforms for generating reagents and therapeutics are needed to detect and control the emergence of new strains, especially early in an outbreak prior to the development of type specific serologic reagents and therapeutics.

The present invention overcomes previous shortcomings in the art by providing methods and compositions comprising a chimeric coronavirus spike protein for treating/and or preventing diseases and disorders caused by infection by a coronavirus.

Tethered protein approach mimicking the intermediate structures of Spike and ACE2 receptor complex as therapeutic and vaccine candidates against SARS-CoV-2

Coronavirus disease 2019 (COVID-19) is caused by the Severe Acute Respiratory Syndrome Coronavirus-2 (SARS-CoV-2) which is a newly emerged human coronavirus. SARS-CoV-2 shares some similarities with SARS-CoV which was shown to be associated with the outbreak in 2002-2003. Due to person-to-person transmission. SARS-CoV-2 has affected more than 118,268,575 confirmed cases and continues to increase globally with high fatality rates. In this regard, SARS-CoV-2 is distinct from SARS-CoV and the underlying reason for this feature is not known. SARS-CoV-2 was initially reported as an agent associated with disease in Wuhan, China. Since then, the virus has spread to Europe. US as well being responsible for a recent and ongoing significant outbreak in 6 continents involving 202 countries. These findings have suggested that the SARS-CoV-2 may have become virulent. Currently, there are no vaccines or therapeutics available to combat SARS-CoV-2 infections. The detection of infected individuals by nucleic acid-based assays followed by isolation and social distancing are the measures used to curb virus infections. This scenario calls for investigations into intervention strategies including antivirals and immunotherapy.

Due to the nature of viral mutations and severity of disease, there is an urgent need to develop novel strategies to control these variants and its pathogenesis, including effective cross-protection vaccines. Additionally, there is a need in the art for effective therapies to treat viral infections caused by these viruses including their mutated forms.

### SUMMARY OF THE INVENTION

In one embodiment, the invention relates to an immunological composition comprising a nucleic acid molecule, wherein the nucleic acid molecule encodes a peptide comprising an amino acid sequence of: a) an amino acid sequence selected from the group consisting of SEQ ID NO:2, SEQ ID NO:4, SEQ ID NO:6, SEQ ID NO:8, SEQ ID NO:12, and an amino acid sequence that is 90% identical or greater to SEQ ID NO:2. SEQ ID NO:4. SEQ ID NO:6, SEQ ID NO:8, or SEQ ID NO:12; b) an immunogenic fragment comprising at least 90% identity over at least 60% of the amino acid sequence of SEQ ID NO:2, SEQ ID NO:4, SEQ ID NO:6, SEQ ID NO:8, or SEQ ID NO:12; or c) an immunogenic fragment comprising least 60% of the amino acid sequence of SEQ ID NO:2, SEQ ID NO:4. SEQ ID NO:6, SEQ ID NO:8, or SEQ ID NO:12.

In one embodiment, the nucleic acid is a DNA molecule. In one embodiment, the nucleic acid is an RNA molecule.

In one embodiment, the nucleic acid molecule comprises a nucleotide sequence selected from the group consisting of: a) a nucleotide sequence selected from the group consisting of SEQ ID NO:1, SEQ ID NO:3, SEQ ID NO:5. SEQ ID NO:7, SEQ ID NO:9, SEQ ID NO:11, and a nucleotide sequence that is 90% identical or greater to SEQ ID NO:1, SEQ ID NO:3, SEQ ID NO:5, SEQ ID NO:7, SEQ ID NO:9, or SEQ ID NO:11; b) an immunogenic fragment comprising at least 90% identity over 60% of the nucleotide sequence of SEQ ID NO:1, SEQ ID NO:3, SEQ ID NO:5, SEQ ID NO:7, SEQ ID NO:9, or SEQ ID NO:11; or c) an immunogenic fragment comprising at least 60% of the nucleotide sequence of SEQ ID NO:1, SEQ ID NO:3. SEQ ID NO:5, SEQ ID NO:7, SEQ ID NO:9, or SEQ ID NO:11.

In one embodiment, the nucleotide sequence is operably linked to at least one regulatory sequence selected from the group consisting of a start codon, an IgE leader sequence, and a stop codon.

In one embodiment, the nucleic acid molecule is an expression vector. In one embodiment, the nucleic acid molecule is a plasmid.

In one embodiment, the nucleic acid molecule is incorporated into a viral particle.

In one embodiment, the immunogenic composition comprises an adjuvant.

In one embodiment, the immunogenic composition comprises a pharmaceutically acceptable excipient.

In one embodiment, the invention relates to a nucleic acid molecule encoding a peptide comprising an amino acid sequence of: a) an amino acid sequence selected from the group consisting of SEQ ID NO:2, SEQ ID NO:4, SEQ ID NO:6, SEQ ID NO:8, SEQ ID NO:12, and an amino acid sequence that is 90% identical or greater to SEQ ID NO:2, SEQ ID NO:4, SEQ ID NO:6, SEQ ID NO:8, or SEQ ID NO:12; b) an immunogenic fragment comprising at least 90% identity over at least 60% of the amino acid sequence of SEQ ID NO:2, SEQ ID NO:4, SEQ ID NO:6, SEQ ID NO:8, or SEQ ID NO:12; or c) an immunogenic fragment comprising least 60% of the amino acid sequence of SEQ ID NO:2, SEQ ID NO:4, SEQ ID NO:6, SEQ ID NO:8, or SEQ ID NO:12.

In one embodiment, the invention relates to a nucleic acid molecule comprising a nucleotide sequence selected from the group consisting of: a) a nucleotide sequence selected from the group consisting of SEQ ID NO:1, SEQ ID NO:3, SEQ ID NO:5, SEQ ID NO:7, SEQ ID NO:9, SEQ ID NO:11, and a nucleotide sequence that is 90% identical or greater to SEQ ID NO:1, SEQ ID NO:3. SEQ ID NO:5, SEQ ID NO:7, SEQ ID NO:9, or SEQ ID NO:11; b) an immunogenic fragment comprising at least 90% identity over 60% of the nucleotide sequence of SEQ ID NO:1, SEQ ID NO:3. SEQ ID NO:5, SEQ ID NO:7, SEQ ID NO:9, or SEQ ID NO:11; or c) an immunogenic fragment comprising at least 60% of the nucleotide sequence of SEQ ID NO:1, SEQ ID NO:3, SEQ ID NO:5, SEQ ID NO:7, SEQ ID NO:9, or SEQ ID NO:11.

In one embodiment, the invention relates to an immunological composition comprising a peptide comprising an amino acid sequence of: a) an amino acid sequence selected from the group consisting of SEQ ID NO:2, SEQ ID NO:4, SEQ ID NO:6, SEQ ID NO:8, SEQ ID NO:12, and an amino acid sequence that is 90% identical or greater to SEQ ID NO:2, SEQ ID NO:4, SEQ ID NO:6, SEQ ID NO:8, or SEQ ID NO:12; b) an immunogenic fragment comprising at least 90% identity over at least 60% of the amino acid sequence of SEQ ID NO:2, SEQ ID NO:4, SEQ ID NO:6, SEQ ID NO:8, or SEQ ID NO:12; or c) an immunogenic fragment comprising least 60% of the amino acid sequence of SEQ ID NO:2. SEQ ID NO:4, SEQ ID NO:6. SEQ ID NO:8, or SEQ ID NO:12.

In one embodiment, the invention relates to a peptide comprising an amino acid sequence of: a) an amino acid sequence selected from the group consisting of SEQ ID NO:2. SEQ ID NO:4, SEQ ID NO:6, SEQ ID NO:8, SEQ ID NO:12, and an amino acid sequence that is 90% identical or greater to SEQ ID NO:2. SEQ ID NO:4, SEQ ID NO:6. SEQ ID NO:8, or SEQ ID NO:12; b) an immunogenic fragment comprising at least 90% identity over at least 60% of the amino acid sequence of SEQ ID NO:2. SEQ ID NO:4, SEQ ID NO:6, SEQ ID NO:8, or SEQ ID NO:12; or c) an immunogenic fragment comprising least 60% of the amino acid sequence of SEQ ID NO:2, SEQ ID NO:4, SEQ ID NO:6, SEQ ID NO:8, or SEQ ID NO:12.

In one embodiment, the invention relates to a method of treating or preventing COVID infection in a subject in need thereof, the method comprising administering an immunological composition of the disclosure to the subject.

In one embodiment, the method comprises electroporation or injection.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1C, depicts representative schematics of DNA sequences and Western blotting of the resulting proteins for expression of Pan-Corona DNA vaccines. FIG. 1A depicts schematic representations of the DNA sequences of the Pan-C1 (SEQ ID NO:1), Pan-C2 (SEQ ID NO:3), and Pan-C3 (SEQ ID NO:5) vaccines or full SARS-CoV-2 Spike and ORF3a protein with a CMV promotor for inclusion in a plasmid. FIG. 1B depicts a representative Western blot of whole-cell lysate of cells that have been transfected with the vaccines, demonstrating expression of the RBD-ACE2 conjugate proteins for Pan-C1 (SEQ ID NO:2), Pan-C2 (SEQ ID NO:4), Pan-C3 (SEQ ID NO:6) and full Spike and ORF3a protein. Immunoblotting for SARS-CoV-2 RBD reveals bands for all proteins at their expected molecular weights. FIG. 1C depicts a representative Western blot of whole-cell lysate of cells that have been transfected with the vaccines, demonstrating expression of the RBD-ACE2 conjugate proteins for Pan-C1 (SEQ ID NO:2). Pan-C2 (SEQ ID NO:4), Pan-C3 (SEQ ID NO:6) and full Spike and ORF3a protein. Immunoblotting for ACE2 reveals bands for all vaccine protein conjugates at there expected molecular weights but no band is observed for the Spike and ORF3a protein.

FIG. 2, comprising FIG. 2A through FIG. 2C, depicts a representative experimental procedure and results of humoral responses of mice to the vaccines. FIG. 2A depicts a representative experimental setup, with injections with vaccine, full Spike protein, or empty vector at days 0 and 21. FIG. 2B depicts representative ELISA assay results of serial dilutions of serum collected on days 0, 21, and 35 for antibodies to RBD-ACE2 (top), RBD (middle), and ACE2 (bottom) at all time points (left), at the 21-day timepoint (middle), and 35-day timepoint (right). FIG. 2C depicts representative quantification of total IgG antibodies binding RBD-ACE2 (top), RBD (middle), and ACE2 (bottom). The novel Pan-CoV2 vaccines elicited considerable humoral responses in mice and exhibited immune reactivity with recombinant SARS-CoV-2 Spike-ACE2, with a significant increase in IgG reactivity between initial and boost immunization. n=7.

FIG. 3 depicts representative ELISA assay results for humoral immune response against variants of SARS-CoV-2. Serum samples taken at days 0, 21, and 35 from mice vaccinated with empty vector (pGLS101), Pan-C1, Pan-C2, and Pan-C3 were serially diluted and used for ELISA assays with wildtype, alpha beta, gamma, delta, and omicron variant RBD proteins. Antibodies produced from vaccinated animals demonstrated diverse binding abilities against the variants tested. n=7.

FIG. 4 depicts representative day 35 results of an ELISA assay for antibodies binding to variants of SARS-CoV-2. Serum samples taken at day 35 from mice vaccinated with empty vector (pGLS101), full Spike protein, Pan-C1, and Pan-C2 were serially diluted and used for ELISA assays with wildtype, alpha beta, gamma, delta, and omicron variant RBD proteins. n=7.

FIG. 5 depicts representative analysis of IgG subclasses produced by mice in response to the vaccines, demonstrating predominantly Th1 responses and IgG class switching. Serum samples taken at days 21 and 35 from mice vaccinated with empty vector (pGLS101), Pan-C1, Pan-C2, or full Spike protein and the ratio of IgG2a to IgG1 determined by ELISA assays.

FIG. 6 depicts representative results of the protective activity of immune sera in Alpha Screen assays. Assays were performed with wildtype, beta, delta, lambda, and omicron Spike proteins and serum collected from mice vaccinated with empty vector (pGLS101), Pan-C1, or Pan-C3. Monoclonal antibodies casirivimab and imdevimab were used as positive controls. n=4.

FIG. 8 depicts representative protective activity of immune sear in vitro, demonstrated by the neutralization curves of wildtype SARS-CoV-2 by Pan-C1 and Pan-C3. Heat-inactivated immune sera from negative control (pGLS101), Pan-C1-, and Pan-C3-treated mice were serially diluted and pre-incubated with wildtype SARS-CoV-2 prior to addition to Vero-E6 cells. After incubation, percent neutralization was determined by viral-mediated cell death compared to uninfected controls. Neutralization curves were inferred by Prism software.

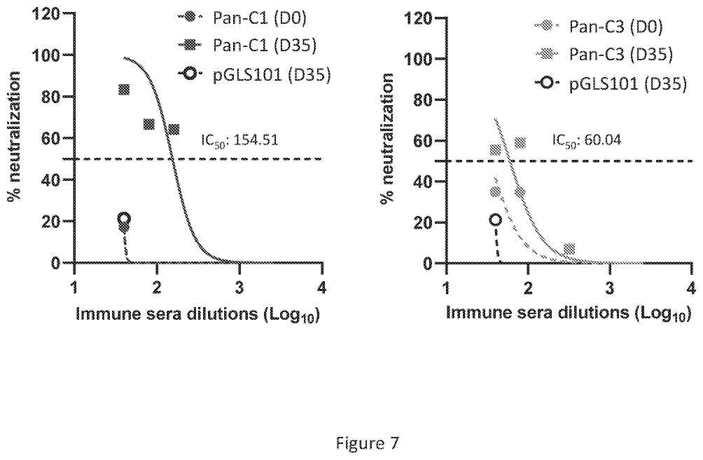

### DETAILED DESCRIPTION

In one aspect, the present invention provides an immunogenic composition targeting a COVID-ACE2 complex (“C-A Complex”) antigen. Further aspects of the present invention are treatments and/or preventions of infection using the disclosed immunogenic composition alone or in combination with additional vaccines or therapeutics.

In another aspect, the present invention provides a method of generating novel antibodies that target SARS-CoV-2 using the C-A Complex antigen and the encoding constructs described herein. Additionally, aspects of the present invention include the resulting synthetic antibodies and the use of same as a treatment for SARS-CoV-2 infections.

The immunogenic composition can be used to protect against and treat SARS infections, in particular SARS-CoV-2. The immunogenic composition can elicit both humoral and cellular immune responses that target the antigen. The immunogenic composition can elicit neutralizing antibodies and immunoglobulin G (IgG) antibodies that are reactive with the C-A Complex antigen. The immunogenic composition can also elicit a CD8⁺ T cell response that is reactive to the antigen and produce one or more of interferon-gamma (IFN-γ) and tumor necrosis factor alpha (TNF-α). In one embodiment, the immunogenic composition can also elicit a CD4⁺ T cell response that is reactive to the antigen and produce one or more of IFN-γ and TNF-α.

In one embodiment, the invention includes a nucleic acid vaccine against SARS-Co-V-2, including one or more mutated versions. In one embodiment, the vaccine comprise a plasmid encoding a C-A Complex antigen. In one embodiment, the C-A Complex antigen further comprise mutations that elicits an immune response in a subject such that the neutralizing capabilities of Tcells and antibodies that form such immune response remain. As an extension of these various aspects of the invention, each C-A Complex antigen can be genetically-optimized, subcloned into modified mammalian expression vectors, and then delivered using one or more techniques to enhance transfection of the nucleic acid constructs, including for example vaccine and antigen version, such as for example in vivo electroporation (EP), suction (negative pressure) manipulation, lipid micelles or lipid based particles, and gold particles.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s).” “include(s).” “having,” “has.” “can,” “contain(s).” and variants thereof, as used herein, are intended to be open-ended transitional phrases, terms, or words that do not preclude the possibility of additional acts or structures. The singular forms “a.” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Adjuvant” as used herein means any molecule added to the immunogenic composition described herein to enhance the immunogenicity of the antigen.

“Antibody” as used herein means an antibody of classes IgG, IgM. IgA. IgD or IgE, or fragments, fragments or derivatives thereof, including Fab, F(ab′)₂, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

“C-A Complex” as used herein means the sequence representing antigenic domains spanning the spike protein and the ACE2 receptor that form during the intermediate structure or complex formed between SARS-CoV-2 and ACE2 receptor. In particular, this C-A Complex is the RBD/RBM domain of the S1 protein of SARS-CoV-2 fused to ACE2, and in some instances include point mutations for existing strains of SAR-CoV-2.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and C-G) or Hoogsteen base pairing between nucleotides or nucleotide analogs of nucleic acid molecules.

As used herein, the term “expressible form” refers to gene constructs that contain the necessary regulatory elements operably linked to a coding sequence that encodes a target protein or an immunomodulating protein, such that when present in the cell of the individual, the coding sequence will be expressed.

“Fragment” as used herein means a nucleotide sequence or a portion thereof that encodes a polypeptide capable of eliciting an immune response in a mammal. The fragments can be DNA fragments selected from at least one of the various nucleotide sequences that encode protein fragments set forth below.

“Fragment” or “immunogenic fragment” with respect to polypeptide sequences means a polypeptide capable of eliciting an immune response in a mammal that cross reacts with a full length endogenous antigen. Fragments of proteins can comprise at least 10%, at least 20%, at least 30%, at least 40%, at least 50%, at least 60%, at least 70%, at least 80%, at least 90% or at least 95% of a protein. In some embodiments, fragments of proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of a protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen.

The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a tumor microenvironment protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that is capable of being administered the immunogenic compositions described herein. The mammal can be, for example, a human, chimpanzee, dog, cat, horse, cow, mouse, or rat.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleotide sequence and a second nucleotide sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of a subject from a disease through means of preventing, suppressing, repressing, or completely eliminating the disease. In one embodiment, preventing the disease involves administering an immunogenic composition of the present invention to a subject prior to onset of the disease. In one embodiment, preventing the disease involves administering an immunogenic composition of the present invention to a subject following a treatment so as to prevent reoccurrence or further progression of the disease. Suppressing the disease involves administering an immunogenic composition of the present invention to a subject after induction of the disease but before its clinical appearance. Repressing the disease involves administering an immunogenic composition of the present invention to a subject after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleotide sequence that is substantially identical over the full length of the full gene sequence or a fragment thereof. The nucleotide sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Description

The protein and nucleic acid constructs provided herein mimic the intermediate structures of the spike protein of SARS-CoV-2 and ACE2 receptor complex—the C-A Complex. Such constructs can be prepared as therapeutic and vaccine candidates against SARS-CoV-2, including one or more mutant variants.

SARS-CoV-2 infection of host cells is orchestrated by the interactions between spike glycoprotein S1 subunit and ACE2 receptor on the cell surface followed by S2 mediating the fusion of viral and cell membranes for virus entry. This interaction leads to intermediate structures that are short-lived during natural viral infection. The present invention relates to compositions and methods useful for targeting this interaction in order to induce an immune response.

The constructs provided herein can act as a prophylactic DNA vaccine targeting the SARS-CoV-2 Spike and ORF3a, by in part inducing protecting neutralizing antibodies. The tethered protein, the C-A Complex, is designed to display spike RBD in association with ACE2. The chimeric protein C-A Complex, upon expression, will lead to a conformation of protein due to RBD-ACE2 interaction and is likely to elicit antibodies that will neutralize many variants of SARS-CoV-2, with a goal of all variants. While the antibodies, induced by the currently approved vaccines, have been shown to protect against the initial pandemic strains of SARS-CoV-2, their efficacies are less against the recently emerged alpha (B.1.1.7), beta (B.1.351), gamma (P1) and delta (B.1.671.2) variants.

While not being limited to the following mechanism of action—current knowledge in the field is that SARS-CoV-2 infection of host cells depends on the interaction between virus spike protein and ACE2 receptor leading to dynamic changes in the spike protein.

There are notable limitations at this step which include: i) All the RBDs present in the trimeric spike protein are not in the “up” orientation which is only accessible to receptor binding; ii) The dynamic changes associated with the interaction between RBD and ACE2 are transient and hence may not be in the horizon of the immune system for generating responses. By tethering RBD to ACE2, as a C-A Complex, the conformation that is transient during infection of cells by the virus is presented. Such a structure can generate broadly neutralizing antibody responses, as this conformation is an essential element during the infection of cells by the virus.

### DNA

The invention provides an optimized sequence encoding a C-A Complex antigen. In one embodiment, the C-A Complex antigen encoded by the optimized sequence is capable of eliciting an immune response in a mammal. In one embodiment, the C-A Complex antigen encoded by the optimized sequence can comprise an epitope(s) that makes it particularly effective as an immunogen against which an immune response can be induced.

The optimized sequence can comprise an immunogenic sequence and/or modification(s) for improved expression. Modification can include codon optimization, RNA optimization, addition of a Kozak sequence for increased translation initiation, and/or the addition of an immunoglobulin leader sequence to increase immunogenicity. The C-A Complex antigen encoded by the optimized sequence can comprise a signal peptide such as an immunoglobulin signal peptide, for example, but not limited to, an immunoglobulin E (IgE) or immunoglobulin (IgG) signal peptide. In some embodiments, the antigen encoded by the optimized sequence can comprise a hemagglutinin (HA) tag. The antigen encoded by the optimized sequence can be designed to elicit stronger cellular and/or humoral immune responses than a corresponding native antigen.

In one embodiment, an optimized encoded C-A Complex antigen is operably linked to one or more regulatory elements. In one embodiment, a regulatory element is a leader sequence. In one embodiment, the optimized DNA sequence operably linked to an IgE leader encoding sequence. In one embodiment, the optimized C-A Complex antigen operably linked to an IgE leader sequence.

In one embodiment, a regulatory element is a start codon, or a fragment or homolog thereof, operably linked to a nucleotide sequence comprising a start codon at the 5′ terminus. In one embodiment, the invention relates to an amino acid sequence as set forth herein, or a fragment or homolog thereof, operably linked to an amino acid encoded by a start codon (e.g., a Methionine) at the N-terminus.

In one embodiment, a regulatory element is at least one stop codon. Therefore, in one embodiment, the invention relates to a nucleic acid sequence as set forth herein, or a fragment or homolog thereof, operably linked to a nucleotide sequence comprising at least one stop codon at the 3′ terminus. In one embodiment, the nucleotide sequence is operably linked to two stop codons to increase the efficiency of translational termination.

In one embodiment, the optimized sequence encoding a C-A Complex antigen can encode a peptide having the amino acid sequence of SEQ ID NO:2, SEQ ID NO:4, SEQ ID NO:6, SEQ ID NO:8. SEQ ID NO:10, or SEQ ID NO:12. In one embodiment, the optimized sequence can have the nucleotide sequence of SEQ ID NO:1, SEQ ID NO:2, or SEQ ID NO:3. In some embodiments, the sequence can be the nucleotide sequence having at least about 96%, 97%, 98%, 99% or 100% identity over an entire length of SEQ ID NO:1, SEQ ID NO:3, SEQ ID NO:5, SEQ ID NO:7, SEQ ID NO:9, or SEQ ID NO:11. In other embodiments, sequence can be the nucleotide sequence that encodes the amino acid sequence having at least about 96%, 97%, 98%, 99%, or 100% identity over an entire length of SEQ ID NO:2, SEQ ID NO:4, SEQ ID NO:6. SEQ ID NO:8, SEQ ID NO:10, or SEQ ID NO:12. In some embodiments, the optimized C-A Complex antigen can be encoded by an RNA that is a transcript from a DNA sequence having at least about 96%, 97%, 98%, 99% or 100% identity over an entire length of SEQ ID NO:1, SEQ ID NO:3, SEQ ID NO:5, SEQ ID NO:7, SEQ ID NO:9, or SEQ ID NO:11. In some embodiments, the optimized C-A Complex antigen can be encoded by an RNA that encodes an amino acid sequence having at least about 96%, 97%, 98%, 99% or 100% identity over an entire length SEQ ID NO:1, SEQ ID NO:3, SEQ ID NO:5. SEQ ID NO:7, SEQ ID NO:9, or SEQ ID NO:11.

The optimized encoded C-A Complex antigen can be a peptide having the amino acid sequence of SEQ ID NO:2, SEQ ID NO:4, SEQ ID NO:6, SEQ ID NO:8. SEQ ID NO:10, or SEQ ID NO:12. In some embodiments, the antigen can have an amino acid sequence having at least about 96%, 97%, 98%, 99%, or 100% identity over an entire length of SEQ ID NO:2. SEQ ID NO:4, SEQ ID NO:6, SEQ ID NO:8, SEQ ID NO:10, or SEQ ID NO:12.

Immunogenic fragments of the C-A Complex described herein can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of the full length of SEQ ID NO:2, SEQ ID NO:4, SEQ ID NO:6, SEQ ID NO:8, SEQ ID NO:10, or SEQ ID NO:12. In some embodiments, immunogenic fragments include a leader sequence, such as for example an immunoglobulin leader, such as the IgE leader. In some embodiments, immunogenic fragments are free of a leader sequence.

In one embodiment, the nucleic acid sequence comprises an RNA sequence encoding a C-A Complex immunogen sequence of SEQ ID NO:2. SEQ ID NO:4, SEQ ID NO:6. SEQ ID NO:8, SEQ ID NO:10, or SEQ ID NO:12. For example, nucleic acids may comprise an RNA sequence encoding one or more of C-A Complex constructs herein, a variant thereof, a fragment thereof or any combination thereof.

Immunogenic fragments of proteins with amino acid sequences homologous to immunogenic fragments of SEQ ID NO:2, SEQ ID NO:4, SEQ ID NO:6, SEQ ID NO:8. SEQ ID NO:10, or SEQ ID NO:12 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:2, SEQ ID NO:4, SEQ ID NO:6, SEQ ID NO:8, SEQ ID NO:10, or SEQ ID NO:12. Some embodiments relate to immunogenic fragments that have 90% homology to the immunogenic fragments of SEQ ID NO:2, SEQ ID NO:4, SEQ ID NO:6, SEQ ID NO:8, SEQ ID NO:10, or SEQ ID NO:12. Some embodiments relate to immunogenic fragments that have 97% homology to the immunogenic fragments of SEQ ID NO:2, SEQ ID NO:4. SEQ ID NO:6, SEQ ID NO:8, SEQ ID NO:10, or SEQ ID NO:12. Some embodiments relate to immunogenic fragments that have 98% homology to the immunogenic fragments of SEQ ID NO:2, SEQ ID NO:4, SEQ ID NO:6, SEQ ID NO:8, SEQ ID NO:10, or SEQ ID NO:12. Some embodiments relate to immunogenic fragments that have 99% homology to the immunogenic fragments of SEQ ID NO:2, SEQ ID NO:4, SEQ ID NO:6, SEQ ID NO:8. SEQ ID NO:10, or SEQ ID NO:12. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:2, SEQ ID NO:4, SEQ ID NO:6, SEQ ID NO:8, SEQ ID NO:10, or SEQ ID NO:12. Immunogenic fragments can be at least 60%, at least 65%, at least 70%, at least 75%, at least 80%, at least 85%, at least 90%, at least 95%, at least 96%, at least 97%, at least 98% or at least 99% of the full length SEQ ID NO:2, SEQ ID NO:4. SEQ ID NO:6, SEQ ID NO:8, SEQ ID NO:10, or SEQ ID NO:12. Immunogenic fragments can be at least 96%, at least 97% at least 98% or at least 99% homologous to fragments of SEQ ID NO:2, SEQ ID NO:4, SEQ ID NO:6. SEQ ID NO:8, SEQ ID NO:10, or SEQ ID NO:12. In some embodiments, immunogenic fragments include sequences that encode a leader sequence, such as for example an immunoglobulin leader, such as the IgE leader. In some embodiments, fragments are free of coding sequences that encode a leader sequence.

### Immunogenic Composition

Provided herein are immunogenic compositions, such as vaccines, comprising an optimized sequence, an optimized encoded antigen, a fragment thereof, a variant thereof, or a combination thereof.

The immunogenic composition can be a DNA vaccine, an RNA vaccine, a peptide vaccine, or a combination vaccine. The vaccine can include an optimized nucleotide sequence encoding an antigen. The nucleotide sequence can be DNA, RNA, cDNA, a variant thereof, a fragment thereof, or a combination thereof. The nucleotide sequence can also include additional sequences that encode linker, leader, or tag sequences that are linked to the antigen by a peptide bond. The peptide vaccine can include an antigen, a variant thereof, a fragment thereof, or a combination thereof. The combination DNA and peptide vaccine can include the above described optimized nucleotide sequence and the encoded antigen.

The vaccine of the present invention can have features required of effective vaccines such as being safe so that the vaccine itself does not cause illness or death; being protective against illness; inducing neutralizing antibody; inducing protective T cell responses; and providing ease of administration, few side effects, biological stability, and low cost per dose.

In one embodiment, the immunogenic composition can be a vaccine. The vaccine can be a vaccine using recombinant vectors to deliver antigen, subunit vaccines, and glycoprotein vaccines, for example, but not limited, the vaccines described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference.

The vaccine of the present invention can have features required of effective vaccines such as being safe so that the vaccine itself does not cause illness or death; being protective against illness; inducing neutralizing antibody; inducing protective T cell responses; and providing ease of administration, few side effects, biological stability, and low cost per dose.

### Immune Response

The immunogenic composition can induce an immune response in the subject administered the composition.

The immunogenic composition can induce a humoral immune response in the subject administered the immunogenic composition. The humoral immune response can be induced in the subject administered the immunogenic composition by about 1.5-fold to about 16-fold, about 2-fold to about 12-fold, or about 3-fold to about 10-fold. The humoral immune response can be induced in the subject administered the immunogenic composition by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered the immunogenic composition or a subject administered a non-optimized C-A Complex antigen.

The humoral immune response induced by the immunogenic composition can include an increased level of neutralizing antibodies associated with the subject administered the immunogenic composition as compared to a subject not administered the immunogenic composition.

The humoral immune response induced by the immunogenic composition can include an increased level of IgG antibodies associated with the subject administered the immunogenic composition as compared to a subject not administered the immunogenic composition. The level of IgG antibody associated with the subject administered the immunogenic composition can be increased by about 1.5-fold to about 16-fold, about 2-fold to about 12-fold, or about 3-fold to about 10-fold as compared to the subject not administered the immunogenic composition. The level of IgG antibody associated with the subject administered the immunogenic composition can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered the immunogenic composition or a subject administered a non-optimized C-A Complex antigen.

The immunogenic composition of the present invention can have features required of effective vaccines such as being safe so the vaccine itself does not cause illness or death; is protective against illness resulting from exposure to live pathogens; induces neutralizing antibody to prevent invasion of cells; induces protective T cells against intracellular pathogens; and provides ease of administration, few side effects, biological stability, and low cost per dose.

The immunogenic composition can further induce an immune response when administered to different tissues such as the muscle or skin. The immunogenic composition can further induce an immune response when administered via electroporation, or injection, or subcutaneously, or intramuscularly.

### Fragments

In one embodiment, the immunogenic fragment is an immunogenic fragment of a full length antigen of the invention. As used herein, an immunogenic fragment is a fragment of a full length nucleic acid or amino acid sequence that can induce an immune response significantly similar to that of the full length sequence. In one embodiment, an immunogenic fragment comprises an immunogenic epitope of a full length sequence. In one embodiment, the immunogenic fragment induces an immune response at least about 0.7-fold, at least about 0.8-fold, at least about 0.9-fold, at least about 1.0-fold, at least about 1.1-fold, at least about 1.2-fold, at least about 1.3-fold, at least about 1.4-fold, at least about 1.5-fold, at least about 2.0-fold or greater than 2.0-fold as compared to the full length sequence.

The immunogenic fragment can induce a humoral immune response in the subject administered the immunogenic fragment. The humoral immune response can be induced in the subject administered the immunogenic fragment by about 1.5-fold to about 16-fold, about 2-fold to about 12-fold, or about 3-fold to about 10-fold. The humoral immune response can be induced in the subject administered the immunogenic fragment by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered immunogenic fragment.

The humoral immune response induced by the immunogenic fragment can include an increased level of IgG antibodies associated with the subject administered the immunogenic fragment as compared to a subject not administered the immunogenic fragment. The level of IgG antibody associated with the subject administered the immunogenic fragment can be increased by about 1.5-fold to about 16-fold, about 2-fold to about 12-fold, or about 3-fold to about 10-fold as compared to the subject not administered the immunogenic fragment. The level of IgG antibody associated with the subject administered the immunogenic fragment can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered the immunogenic fragment.

The immunogenic fragment of the present invention can have features required of effective vaccines such as being safe so the vaccine itself does not cause illness or death; is protective against illness resulting from exposure to live viruses; induces neutralizing antibody to prevent invasion of cells; induces protective T cells against intracellular pathogens; and provides ease of administration, few side effects, biological stability, and low cost per dose.

The immunogenic fragment can further induce an immune response when administered to different tissues such as the muscle or skin. The immunogenic fragment can further induce an immune response when administered via electroporation, or injection, or subcutaneously, or intramuscularly.

### Vector

SEQ ID NO:1, SEQ ID NO:3, SEQ ID NO:5, SEQ ID NO:7, SEQ ID NO:9, or SEQ ID NO:11 can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure or be incorporated within the host's genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

### Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

### Plasmid

The one or more vectors can be a plasmid. The plasmid may be useful for transfecting cells with the recombinant nucleic acid construct. The plasmid may be useful for introducing the recombinant nucleic acid construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego. Calif.), which may be used for protein production in Escherichia coli (E. coli). The plasmid may also be pYES2 (Invitrogen. San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen. San Diego, Calif.), which may be used for protein production in mammalian cells such as Chinese hamster ovary (CHO) cells.

### RNA

In one embodiment, the nucleic acid is an RNA molecule. In one embodiment, the RNA molecule is transcribed from a DNA sequence of SEQ ID NO:1, SEQ ID NO:3, SEQ ID NO:5, SEQ ID NO:7, SEQ ID NO:9, or SEQ ID NO: 11. For example, in some embodiments, the RNA molecule is encoded by a DNA sequence at least 90% homologous to SEQ ID NO:1. SEQ ID NO:3, SEQ ID NO:5, SEQ ID NO:7, SEQ ID NO:9, SEQ ID NO:11, or a variant or fragment thereof. In another embodiment, the nucleotide sequence comprises an RNA sequence transcribed by a DNA sequence encoding a polypeptide sequence at least 90% homologous to SEQ ID NO:1. SEQ ID NO:3. SEQ ID NO:5, SEQ ID NO:7, SEQ ID NO:9, SEQ ID NO:11, or a variant or fragment thereof. Accordingly, in one embodiment, the invention provides an RNA molecule encoding one or more of the C-A Complex antigens of SEQ ID NO:2, SEQ ID NO:4, SEQ ID NO:6, SEQ ID NO:8, SEQ ID NO:10, or SEQ ID NO:12. The RNA may be plus-stranded. Accordingly, in some embodiments, the RNA molecule can be translated by cells without needing any intervening replication steps such as reverse transcription. An RNA molecule useful with the invention may have a 5′ cap (e.g. a 7-methylguanosine). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. An RNA molecule may have a 3′ poly-A tail. It may also include a poly-A polymerase recognition sequence (e.g. AAUAAA) near its 3′ end. An RNA molecule useful with the invention may be single-stranded. An RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively. UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### Circular and Linear Vectors

The vector may be a circular plasmid, which may transform a target cell by integration into the cellular genome or exist extrachromosomally (e.g., autonomous replicating plasmid with an origin of replication).

The vector can be pVAX, pcDNA3.0, or provax, or any other expression vector capable of expressing DNA encoding the antigen and enabling a cell to translate the sequence to an antigen that is recognized by the immune system.

Also provided herein is a linear nucleic acid immunogenic composition, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, and/or a polyadenylation signal. The expression of the antigen may be controlled by the promoter. The LEC may not contain any antibiotic resistance genes and/or a phosphate backbone. The LEC may not contain other nucleotide sequences unrelated to the desired antigen gene expression.

The LEC may be derived from any plasmid capable of being linearized. The plasmid may be capable of expressing the antigen. The plasmid can be pNP (Puerto Rico/34) or pM2 (New Caledonia/99). The plasmid may be WLV009, pVAX, pcDNA3.0, or provax, or any other expression vector capable of expressing DNA encoding the antigen and enabling a cell to translate the sequence to an antigen that is recognized by the immune system.

The LEC can be pcrM2. The LEC can be pcrNP, pcrNP and pcrMR can be derived from pNP (Puerto Rico/34) and pM2 (New Caledonia/99), respectively.

### Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleotide sequence encoding the antigen and signals required for efficient polyadenylation of the transcript, ribosome binding sites, and translation termination. The promoter may be a CMV promoter, SV40 early promoter. SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

### Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See. e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### Method of Preparing the Vector

Provided herein is a method for preparing the one or more vectors in which the recombinant nucleic acid construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described elsewhere herein in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### Multiple Vectors

The immunogenic composition may comprise a plurality of copies of a single nucleic acid molecule such a single plasmid, or a plurality of copies of two or more different nucleic acid molecules such as two or more different plasmids. For example, an immunogenic composition may comprise a plurality of two, three, four, five, six, seven, eight, nine or ten or more different nucleic acid molecules. Such compositions may comprise a plurality of two, three, four, five, six, or more different plasmids.

Immunogenic compositions may comprise nucleic acid molecules, such as plasmids, that collectively contain coding sequence for a C-A Complex antigen of SEQ ID NO:2, SEQ ID NO:4, SEQ ID NO:6, SEQ ID NO:8, SEQ ID NO:10, or SEQ ID NO:12. Immunogenic compositions may comprise nucleic acid molecules, such as plasmids, that collectively contain coding sequence for multiple antigens. In one embodiment, the antigens are a C-A Complex antigen and one or more additional cancer antigen. Immunogenic compositions may comprise nucleic acid molecules, such as plasmids, that collectively contain coding sequence for one or more antigen and one or more cancer antigen.

### Methods

Provided herein are methods of treating, protecting against, and/or preventing a C-A Complex associated disease in a subject in need thereof by administering one or more immunogenic composition described herein to the subject. Administration of the immunogenic composition to the subject can induce or elicit an immune response in the subject.

Provided herein is a method for delivering the immunogenic composition for providing genetic constructs and proteins of the C-A Complex antigen which comprise epitopes that make them particular effective against coronavirus, particularly SARS-CoV-2 or COVID. The method of delivering the immunogenic composition or vaccination may be provided to induce a therapeutic and prophylactic immune response. The vaccination process may generate in the mammal an immune response against SARS-CoV-2. The immunogenic composition may be delivered to an individual to modulate the activity of the mammal's immune system and enhance the immune response. The delivery of the immunogenic composition may be the transfection of the antigen as a nucleic acid molecule that is expressed in the cell and delivered to the surface of the cell upon which the immune system recognized and induces a cellular, humoral, or cellular and humoral response. The delivery of the immunogenic composition may be used to induce or elicit and immune response in mammals against SARS-CoV-2 by administering to the mammals the immunogenic composition as discussed above.

Upon delivery of the immunogenic composition and plasmid into the cells of the mammal, the transfected cells will express and secrete antigens for each of the plasmids injected from the immunogenic composition. These proteins will be recognized as foreign by the immune system and antibodies will be made against them. These antibodies will be maintained by the immune system and allow for an effective response.

The immunogenic composition may be administered to a mammal to elicit an immune response in a mammal. The mammal may be human, primate, non-human primate, cow, cattle, sheep, goat, antelope, bison, water buffalo, bison, bovids, deer, hedgehogs, elephants, llama, alpaca, mice, rats, and chicken.

The induced immune response can include an induced humoral immune response and/or an induced cellular immune response. The humoral immune response can be induced by about 1.5-fold to about 16-fold, about 2-fold to about 12-fold, or about 3-fold to about 10-fold. The induced cellular immune response can include a CD8⁺ T cell response, which is induced by about 2-fold to about 30-fold, about 3-fold to about 25-fold, or about 4-fold to about 20-fold.

The immunogenic composition dose can be between 1 μg to 10 mg active component/kg body weight/time and 20 μg to 10 mg component/kg body weight/time. The immunogenic composition can be administered every 1, 2, 3, 4, 5, 6, 7, 8, 9, 10, 11, 12, 13, 14, 15, 16, 17, 18, 19.20, 21, 22, 23, 24, 25, 26, 27, 28, 29, 30, or 31 days. The number of immunogenic composition doses for effective treatment can be 1, 2, 3, 4, 5, 6, 7, 8, 9, or 10.

The immunogenic composition can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration.

The immunogenic composition can be administered prophylactically or therapeutically. In prophylactic administration, the immunogenic compositions can be administered in an amount sufficient to induce an immune response. In therapeutic applications, the immunogenic compositions are administered to a subject in need thereof in an amount sufficient to elicit a therapeutic effect. An amount adequate to accomplish this is defined as “therapeutically effective dose.” Amounts effective for this use will depend on, e.g., the particular composition of the immunogenic composition regimen administered, the manner of administration, the stage and severity of the disease, the general state of health of the subject, and the judgment of the prescribing physician.

The immunogenic composition can be administered by methods well known in the art as described in Donnelly et al. (Ann. Rev. Immunol. 15:617-648 (1997)); Feigner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The nucleic acid of the immunogenic composition can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The immunogenic composition can be delivered via a variety of routes. Typical delivery routes include parenteral administration, e.g., intradermal, intramuscular or subcutaneous delivery. Other routes include oral administration, intranasal, and intravaginal routes. For the nucleic acid of the immunogenic composition in particular, the immunogenic composition can be delivered to the interstitial spaces of tissues of an individual (Feigner et al., U.S. Pat. Nos. 5,580,859 and 5,703,055, the contents of all of which are incorporated herein by reference in their entirety). The immunogenic composition can also be administered to muscle, or can be administered via intradermal or subcutaneous injections, or transdermally, such as by iontophoresis. Epidermal administration of the immunogenic composition can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The immunogenic composition can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the immunogenic composition.

The immunogenic composition can be a liquid preparation such as a suspension, syrup or elixir. The immunogenic composition can also be a preparation for parenteral, subcutaneous, intradermal, intramuscular or intravenous administration (e.g., injectable administration), such as a sterile suspension or emulsion.

The immunogenic composition can be incorporated into liposomes, microspheres or other polymer matrices (Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

### Routes of Administration

The vaccine or pharmaceutical composition can be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, and intraarticular or combinations thereof.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken. For veterinary use, the composition can be administered as a suitably acceptable formulation in accordance with normal veterinary practice. The veterinarian can readily determine the dosing regimen and route of administration that is most appropriate for a particular animal.

The vaccine can be administered by traditional syringes, needleless injection devices, “microprojectile bombardment gene guns”, or other physical methods such as electroporation (“EP”), “hydrodynamic method”, or ultrasound.

The immunogenic composition can be administering to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The one or more cancer antigens of the vaccine can be administered via DNA injection and along with in vivo electroporation.

The immunogenic composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The immunogenic composition may be administered by traditional syringes, needleless injection devices, “microprojectile bombardment gone guns”, or other physical methods such as electroporation (“EP”), suction-based cutaneous delivery method, “hydrodynamic method”, or ultrasound.

The immunogenic composition may be delivered to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The C-A Complex antigen may be delivered via DNA injection and along with in vivo electroporation.

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. Transfection facilitating agents include, but are not limited to a polyanion, polycation, including poly-L-glutamate (LGS), or lipid. In one embodiment, the transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS). Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. In some embodiments, the concentration of the transfection agent in the composition is less than 4 mg/ml, less than 2 mg/mI, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

The present invention also relates to a method of delivering the composition to the subject in need thereof. The method of delivery can include, administering the composition to the subject. In some embodiments, the present invention relates to administration of a nucleic acid molecule to a subject in need thereof. In some embodiments, the nucleic acid molecule is a DNA molecule. In some embodiments, the nucleic acid molecule is an RNA molecule. In some embodiments, the nucleic acid molecule is an mRNA molecule.

Exemplary delivery vehicles include, but are not limited to, microspheres, microparticles, nanoparticles, polymerosomes, liposomes, and micelles. For example, in some embodiments, the delivery vehicle is a lipid nanoparticle loaded with a nucleic acid molecule. Administration can include, but is not limited to, intravenous delivery of a nucleic acid molecule, injection, liposome mediated delivery, lipid micelle mediated delivery, and nanoparticle facilitated delivery.

In some embodiments, the delivery vehicle provides for controlled release, delayed release, or continual release of its loaded cargo. In some embodiments, the delivery vehicle comprises a targeting moiety that targets the delivery vehicle to a treatment site.

In certain instances, expressing a protein by delivering the encoding mRNA has many benefits over methods that use protein, plasmid DNA or viral vectors. During mRNA transfection, the coding sequence of the desirnd protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 min of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunogenic means (ELISAs and Western blots) or by assays described herein to identify agents falling within the scope of the invention.

Administration of the immunogenic composition via electroporation may be accomplished using electroporation devices that can be configured to deliver to a desired tissue of a mammal a pulse of energy effective to cause reversible pores to form in cell membranes allowing penetration of a nucleic acid molecule into the cell.

Administration of the immunogenic composition via a suction-based cutaneous delivery method may be accomplished using a device as described in Lallow et al., 2021, Science Advances, 7(45):1-9.

### Generation of Antigens In Vitro and Ex Vivo

In one embodiment, the optimized C-A Complex antigen is generated in vitro or ex vivo. For example, in one embodiment, a nucleic acid encoding an optimized C-A Complex antigen can be introduced and expressed in an in vitro or ex vivo cell. In one embodiment, the nucleic acid encodes a C-A Complex antigen of SEQ ID NO:2, SEQ ID NO: 4, or SEQ ID NO:6.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual. Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that arm not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### Example 1: Design and Expression of CoV-2-ACE Complex DNA Vaccines

SARS-CoV-2 infection of host cells is orchestrated by the interactions between spike glycoprotein S1 subunit and ACE2 receptor on the cell surface followed by S2 mediating the fusion of viral and cell membranes for virus entry. This interaction leads to intermediate structures that are short-lived during natural viral infection. These intermediate structures present a novel tethered molecule platform with the potential to elicit broadly neutralizing antibodies in addition to T cell responses.

Traditional approaches to preparation of antibodies for the interaction between SARS-CoV-2 and ACE-2 have several problems: 1) all receptor binding domains (RBDs) may not be in an “up” orientation, which is only accessible to receptor binding; 2) the dynamic changes associated with the interactions of the RBD and ACD-2 are transient and not readily accessible for generating immune responses. Therefore, in order to design a vaccine targeting the intermediate stage of CoV-2 entry into a cell, a series of CoV-2 Spike RBD peptides were envisioned to be tethered to an ACE2 protein (C-A). It was reasoned that the tethering of the RBD and ACE2 would mimic the transition state that transiently occurs when SARS-CoV-2 infects a cell, thereby allowing production of antibodies against the target. This approach also allows for the generation of monomeric, dimeric, and trimeric molecules which can induce B- and T-cell immune responses.

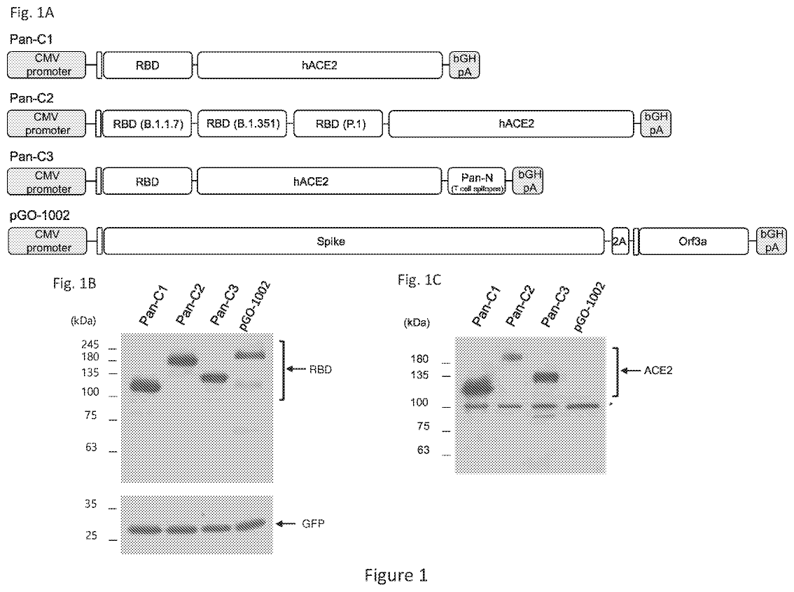

To this end, a series of DNA constructs were prepared. The first DNA construct, comprising SEQ ID NO:1 (FIG. 1A), encodes a chimeric protein of SEQ ID NO:2, comprising a SARS-CoV-2 wildtype RBD tethered to a humane ACE2 protein by a short linker peptide.

A second construct, comprising SEQ ID NO:3, encodes a chimeric protein of SEQ ID NO:4 (FIG. 1A), which comprises the RBD of the alpha variant (B.1.1.7), the beta variant (B.1.351), and the gamma variant (P1) tethered to each other in series via short linker peptides and tethered to human ACE2 by a third linker peptide.

In order to prove T-cell response for viral clearance, the third construct, comprising SEQ ID NO:5 (FIG. 1), encodes a chimeric protein of SEQ ID NO:6, which comprises the wildtype RBD tethered to human ACE2 as in SEQ ID NO:2 further tethered to N⁺ epitopes.

For comparison, a construct comprising the full Spike and ORF3a proteins (pGO-1002) was used as a positive control. All constructs were expressed in mammalian cell culture. Cell lysates were run on 5-15% SDS gels followed by immunoblotting for SARS-CoV-2 RBD (FIG. 1B) or ACE2 (FIG. 1C). As can be seen, all constructs were positive for RBD but only the conjugates were positive for ACE2 (FIGS. 1B and 1C). All protein bands were present at their expected molecular weights.

### Example 2: Humoral Responses of C-A Complex DNA Vaccines in Balb/c Mice

### Induction of Immune Response

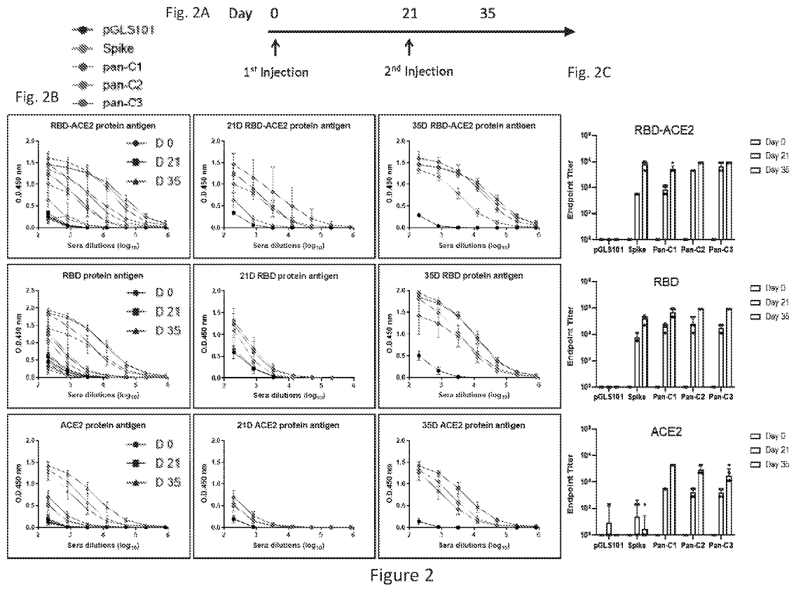

Given the successful expression of the C-A complexes, mice Balb/c mice were inoculated on day 0 and day 21 with DNA constructs of SEQ ID NOs:1, 3, or 5, a construct encoding the full Spike protein (pGO-1002) as a positive control, or an empty vector (pGLS101) as a negative control (FIG. 2A). Sera from the mice (n=5) was collected on day 0, day 21, and day 35. Elisa assays were performed on serial dilutions (200-, 800-, 3200-, 12800-, 51200-, 204800-, and 819200-fold; n=7) of the sera to measure the humoral immune responses against SARS-CoV-2-ACE2 recombinant protein (FIG. 2B). Final serum antibody titers were determined for antibodies specific to RBD-ACE2, RBD, and ACE2 (FIG. 2C).

### Humoral Immune Response Against Viral Variants

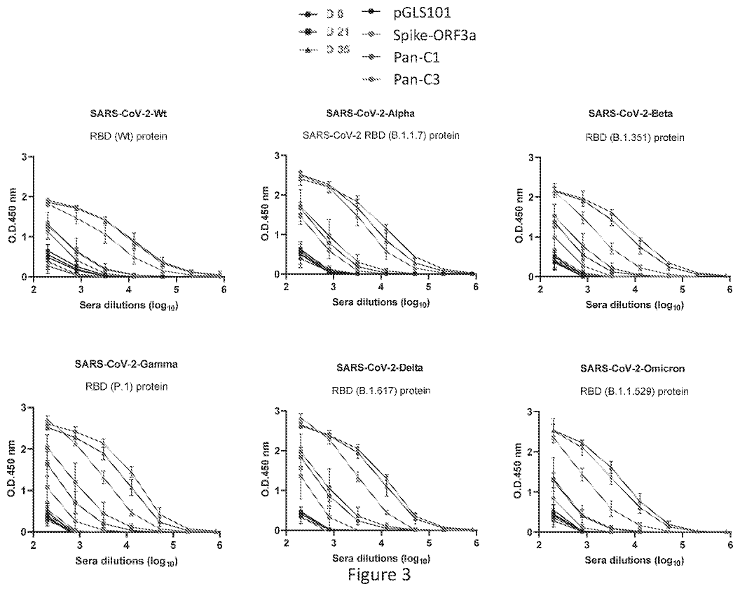

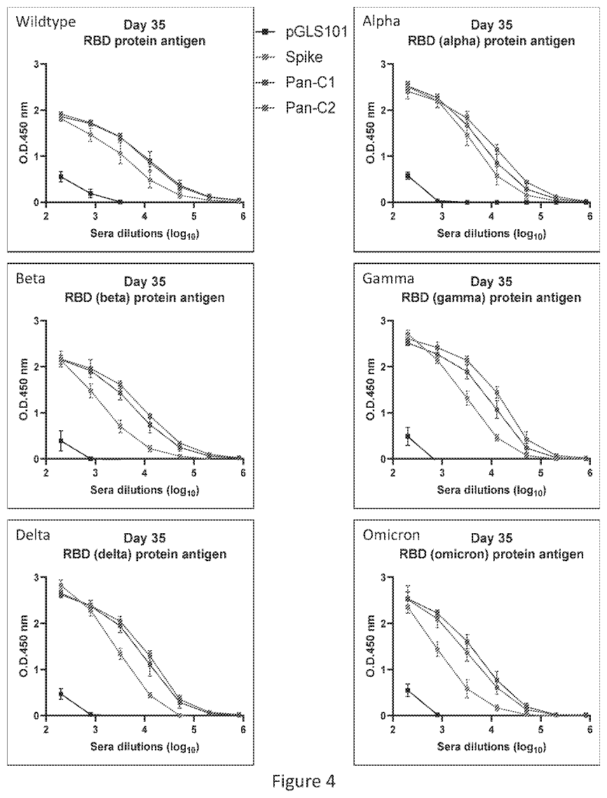

To examine the efficacy of the vaccine against variants of SARS-CoV-2, the sera collected from the animals on days 0, 21, and 35 in Example 2 were tested against wildtype (Wt), alpha, beta, gamma, delta, and omicron variants of SARS-CoV-2, with similar responses against all variants (FIG. 3). Endpoint results (day 35) are repeated in FIG. 4 for clarity.

### IgG Class Switching in Immune Response

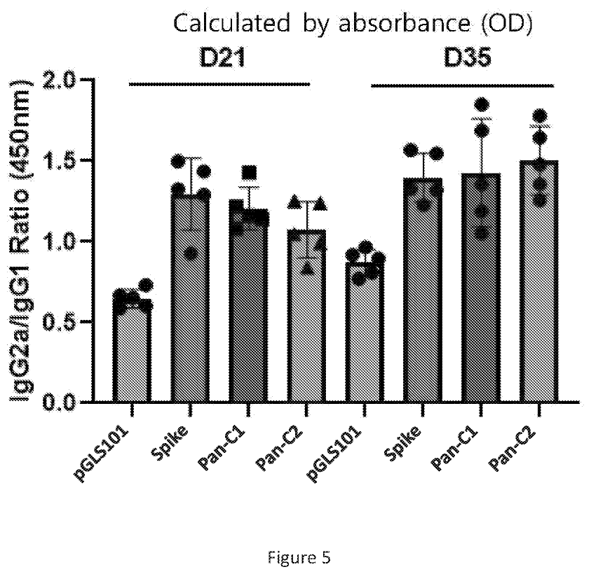

For a more detailed look at the humoral response the IgG subclasses were analyzed. Antibodies from the serum taken on days 21 and 35 were examined, and mostly Th1 responses were observed. By day 31 an increase in the ratio of IgG2a to IgG1 was apparent (FIG. 5).

### HTRF Assay for Inhibition of CoV-Spike Binding to ACE2

To examine the efficacy of antibodies produced in response to the vaccines, homogenous time resolved fluorescence (HTRF) assays were performed on the serum samples previously collected. Experimental serum samples (pGLS101 day 0 and day 35: pGO-1101 day 0 and day 35 and pGO1103 day 0 and day 35) were serially diluted 1:2 in assay buffer (25 mM Tris, pH 7.4, 150 mM KCl, 0.05% CHAPS, 0.1% BSA), and dilutions were pre-incubated with 2 nM HIS-CoV-Spike RBD (either wild-type, beta, delta, lambda and omicron variants, Sino Biological) prebound to 600 ng/mL anti-HIS-d2 HTRF acceptor (PerkinElmer) in a total volume of 10 μL of assay buffer in white, low-volume 384 well plates. After one hour, assays were initiated by adding 5 μL of 6 nM biotin-ACE2 (2 nM final concentration, Acros BioSciences) prebound to 50 ng/mL streptavidin-terbium HTRF donor (PerkinElmer). After an additional 2 hour incubation, HTRF signals were measured using a ClarioStar plate reader (BMG LabTech) at 320 nm excitation and 620/665 nm emission with a 50 μs delay and window time of 200 μs. The raw data at each wavelength were then converted to the HTRF ratio by RFU 665/RFU 620×10000. Ratio values were then converted to percent inhibition, where 0% was equal to the HTRF value in the absence of plasma and 100% was equal to the HTRF value in the absence of HIS-CoV-Spike RBD.

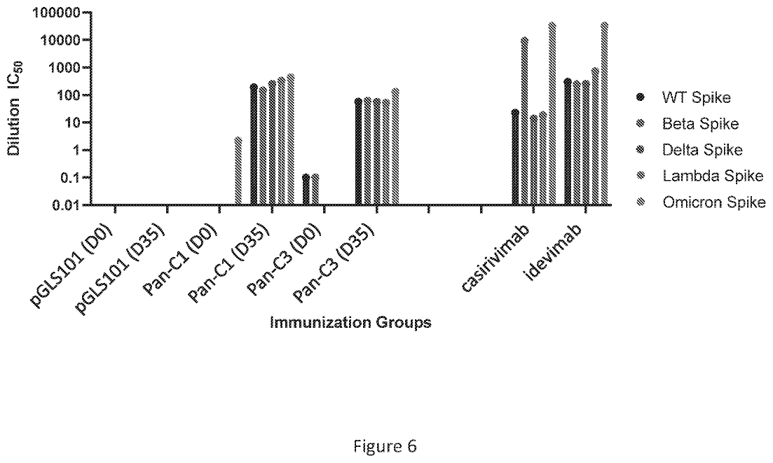

To calculate IC50 values, percent inhibition values were fit to 4-parameter dose-response curves using the dilution factor as the X coordinate, the top parameter was fixed to 100% and the slope constrained between 1-2 (FIG. 6). Ab-933 and Ab-987 (REGEN-COV (casirivimab with imdevimab also known as REGN10933 and REGN10987) MAbs were obtained from commercial sources.

### Protective Activity of Sera in In Vitro Live Virus Mays

Heat-inactivated immune sera from pGLS-101, pGO-1101, and pGO-1103 immunized mice (day 0 and day 35) were serially diluted and pre-incubated with wildtype SARS-CoV-2 before addition to Vero-E6 cell cultures. After a 72-hour incubation, the percent neutralization was determined by viral mediated cells death assay in infected cells compared with controls.

Normalized percentage neutralization values are plotted against the logarithm of the immune sera dilution factors, and neutralization curves are inferred by the Prism software (FIG. 7). The neutralization curves demonstrate that pGO-1101 immune serum possesses strong neutralization capability against SARS-CoV-2 Wt. isolates from the first outbreak.

TABLE 1
Sequence Listing Information

| | | DNA SEQ | AA SEQ | Trimmed DNA | Trimmed AA |
|---|---|---|---|---|---|
| Vaccine | Regions | ID NO | ID NO | SEQ ID NO | SEQ ID NO |
| Pan-C1 | wtRBD-hACE2 | 1 | 2 | 7 | 8 |
| Pan-C2 | αRBD-βRBD-γRBD-hACE2 | 3 | 4 | 9 | 10 |
| Pan-C3 | wtRBD-hACE2-PanN | 5 | 6 | 11 | 12 |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. An immunogenic composition comprising a nucleotide sequence encoding a C-A Complex antigen comprising an antigenic domain spanning a spike protein of SARS-CoV-2 and an ACE2 receptor of an intermediate structure formed between SARS-CoV-2 and ACE2 receptor.

2. The composition of claim 1, wherein the spike protein component of the antigenic domain is an RBD/RBM domain of S1 protein of SARS-CoV-2.

3. The composition of claim 1, wherein the spike protein component includes point mutations identified in evolved strains of SAR-CoV-2.

4. The composition of claim 2, wherein the C-A Complex antigen comprises an amino acid sequence selected from the group consisting of SEQ ID NO:2, SEQ ID NO:4, SEQ ID NO:6, SEQ ID NO:8, SEQ ID NO:10, SEQ ID NO:12, and an amino acid sequence that is 90% identical or greater to SEQ ID NO:2, SEQ ID NO:4, SEQ ID NO:6, SEQ ID NO:8, SEQ ID NO:10, or SEQ ID NO:12.

5. The composition of claim 4, wherein the nucleic acid molecule is selected from the group consisting of a DNA molecule and an RNA molecule.

6. The composition of claim 5, wherein the nucleotide sequence comprises one or more plasmids.

7. The composition of claim 1, further comprising a nucleotide sequence encoding an adjuvant.

8. The composition of claim 1, wherein a nucleotide sequence encoding region is operably linked to at least one regulatory sequence selected from the group consisting of a start codon, an IgE leader sequence and a stop codon.

9. The composition of claim 1, wherein the nucleic acid molecule is incorporated into a viral particle.

10. The composition of claim 1, further comprising a pharmaceutically acceptable excipient.

11. A method of treating or preventing COVID infection in a subject in need thereof, the method comprising administering the composition of claim 1 to the subject.

12. The method of claim 11, wherein the administering step comprises electroporation.

13. A nucleic acid molecule comprising one or more nucleotide sequences selected from the group consisting of: SEQ ID NO:1, SEQ ID NO:3, SEQ ID NO:5, SEQ ID NO:7, SEQ ID NO:9, SEQ ID NO:11, and a nucleotide sequence that is 90% identical or greater to SEQ ID NO:1, SEQ ID NO:3, SEQ ID NO:5, SEQ ID NO:7, SEQ ID NO:9, or SEQ ID NO:11.

14. A protein comprising one or more amino acid sequences selected from the group consisting of: SEQ ID NO:2, SEQ ID NO:4, SEQ ID NO:6, SEQ ID NO:8, SEQ ID NO:10, SEQ ID NO:12, and an amino acid sequence that is 90% identical or greater to SEQ ID NO:2, SEQ ID NO:4, SEQ ID NO:6, SEQ ID NO:8, SEQ ID NO:10, or SEQ ID NO:12.
